(19) United States
(12) Plant Patent Application Publication
(10) Pub. No.: US 2005/0155122 P1
(43) Pub. Date: Jul. 14, 2005

(54) Strawberry plant and fruit named "La Cle des Champs"

(76) Inventor: Shahrokh Khanizadeh, Baie d'Urfe (CA)

Correspondence Address: CANTOR COLBURN, LLP, 55 GRIFFIN ROAD SOUTH, BLOOMFIELD, CT, 06002, US

(21) Appl. No.: 10/897,717

(22) Filed: Jul. 23, 2004

(30) Foreign Application Priority Data
Jul. 23, 2003 (CA) 03-3781

(51) Int. Cl. A01H 5/00; A01H 1/00; C12N 15/82; C12N 15/87

(52) U.S. Cl. PLT/208

(57) ABSTRACT

The present invention relates to a new and distinct variety of strawberry named “La Cle des Champs”. The variety is botanically identified as Fragaria×ananassa. The new variety is distinguished from other varieties by a number of properties including but not limited to: shape and lengthy leaves; fruit color and texture; and disease and temperature hardiness.

Latin name of the genus and species of the plant claimed: The variety is botanically identified as Fragaria×ananassa.

### BACKGROUND OF THE INVENTION

The new variety “La Cle des Champs” originated from the controlled cross of the strawberry plants SJ89244-6E and SJ8518-11 made in 1993. Evaluations have been ongoing since 1994. Selection criteria include winter hardiness, fruit quality, shelf life and disease resistance. “La Cle des Champs” were planted in 2002 at l'Acadie, Québec. The plots were grown in matted rows approximately 2.5 m in length, and consisted of four replicates per variety. Evaluations were made on plants during 2002-2003 growing seasons.

### SUMMARY OF THE INVENTION

The present invention relates to a new and distinct variety of strawberry named “La Cle des Champs”. The variety is botanically identified as Fragaria×ananassa. The new variety is distinguished from other varieties by a number of characteristics as set forth below.

### COMPARISON TO SIMILAR VARIETIES

The low temperature tolerance of “La Cle des Champs” is high whereas in Jewel it is medium. The terminal leaflets of “La Cle des Champs” are longer than they are broad whereas they are as long as they are broad in Jewel. The leaves of “La Cle des Champs” have weak interveinal blistering whereas it is very weak in Jewel. “La Cle des Champs” is resistant to powdery mildew (Sphaerotheca macularis Wallr. Ex Fr.) and leaf scorch (Diplocarpon earlina Ell. & Ev.), whereas Jewel is moderately resistant. “La Cle des Champs” is moderately susceptible to susceptible to Verticillium wilt whereas “Jewel” is moderately susceptible. “La Cle des Champs” is moderately susceptible to susceptible to leaf spots (Mycosphaerella fragariae (Tul. Lindau) whereas Jewel is moderately resistant to moderately susceptible.

### BRIEF DESCRIPTION OF THE DRAWINGS

The accompanying photographs show typical specimens of the new variety, including fruit, foliage and flowers, in color as nearly true as it is reasonably possible to make in color illustrations of these characteristics.

FIG. 1 illustrates the upper side of the flowers and the plant.

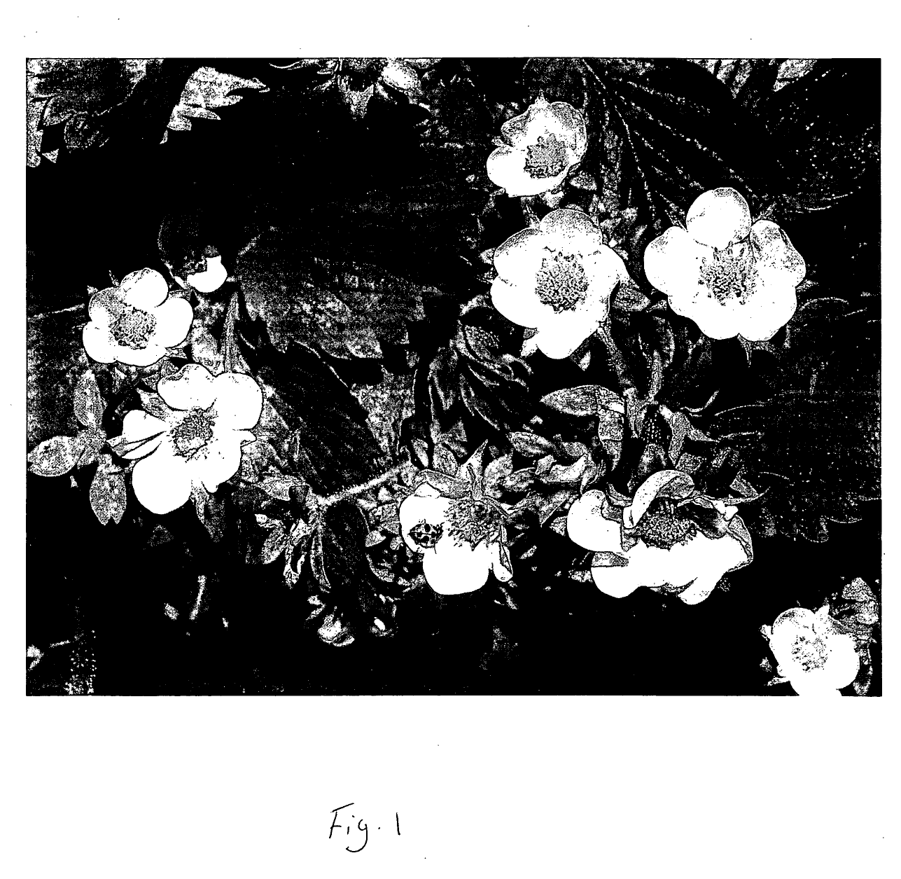

FIG. 2 illustrates the leaves of the plant.

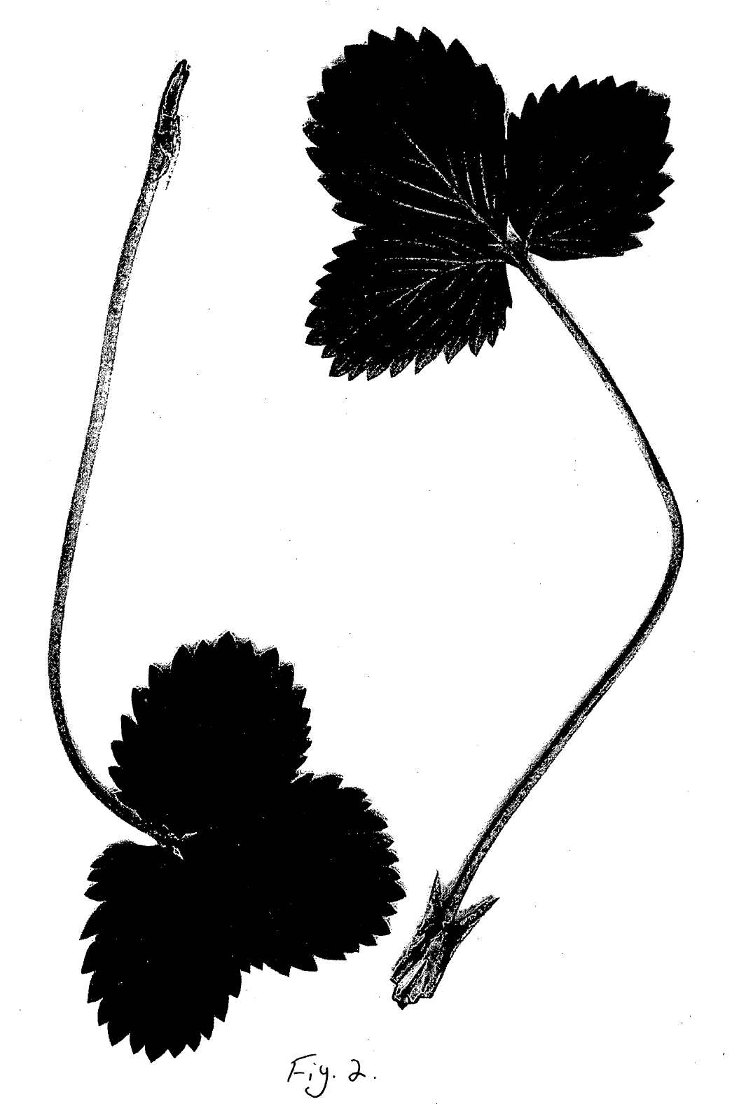

FIG. 3 illustrates the leaves of the plant.

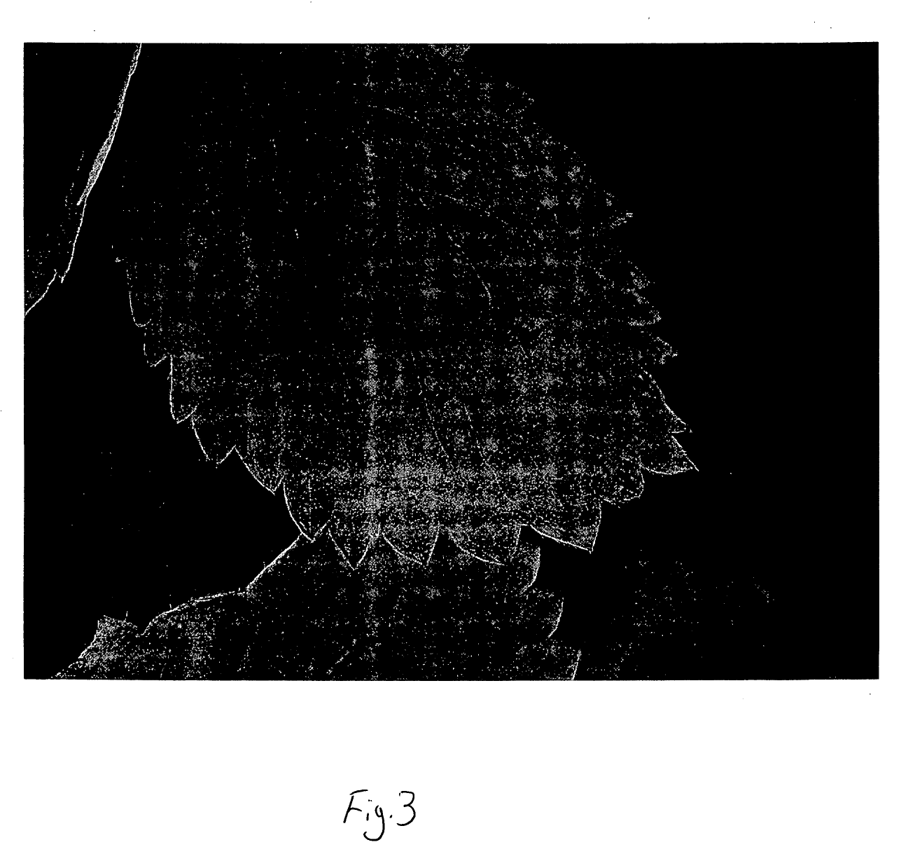

FIG. 4 illustrates the under side of the flowers.

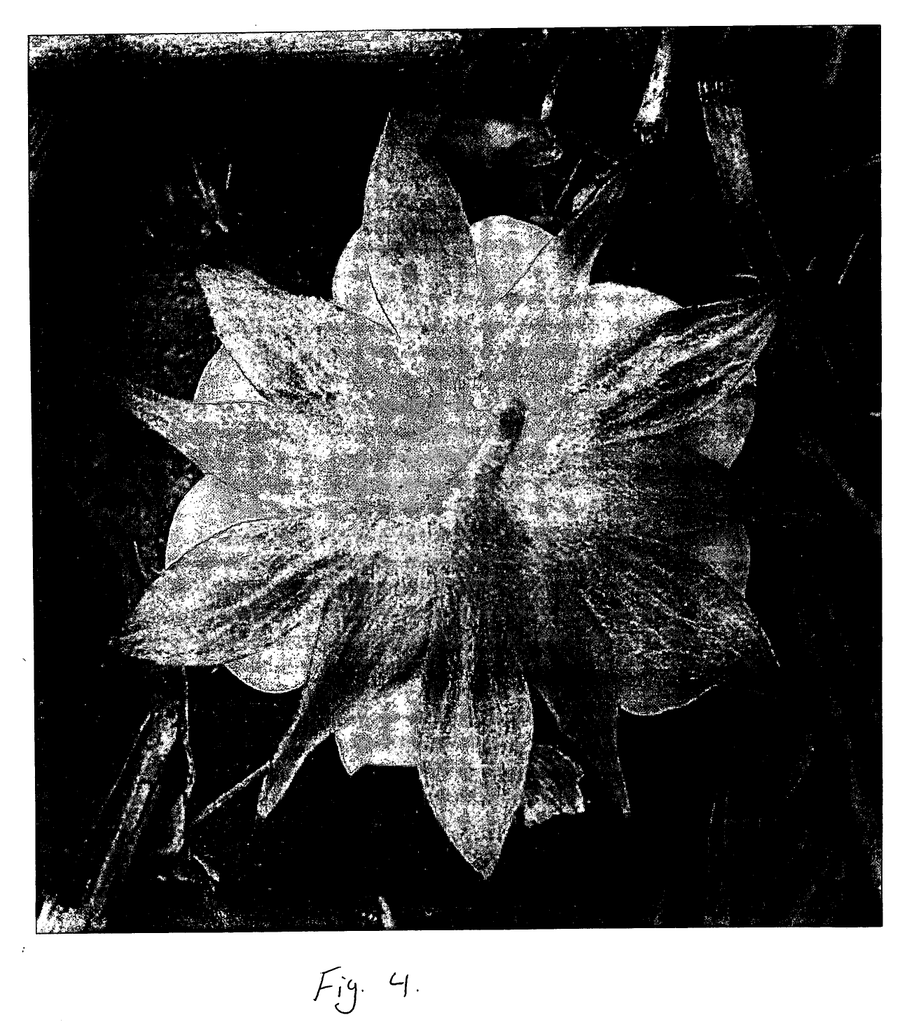

FIG. 5 illustrates a close-up of the fruit.

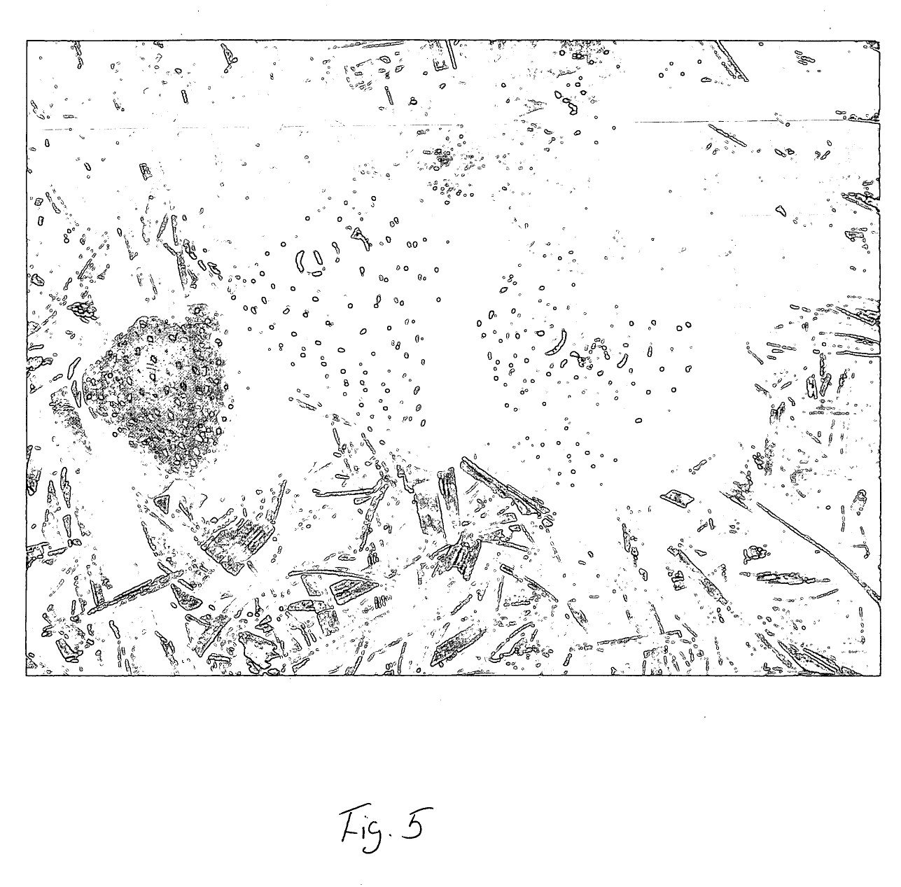

FIG. 6 illustrates the fruit in longitudinal and transversal cross-sections and upper side and side of the fruit.

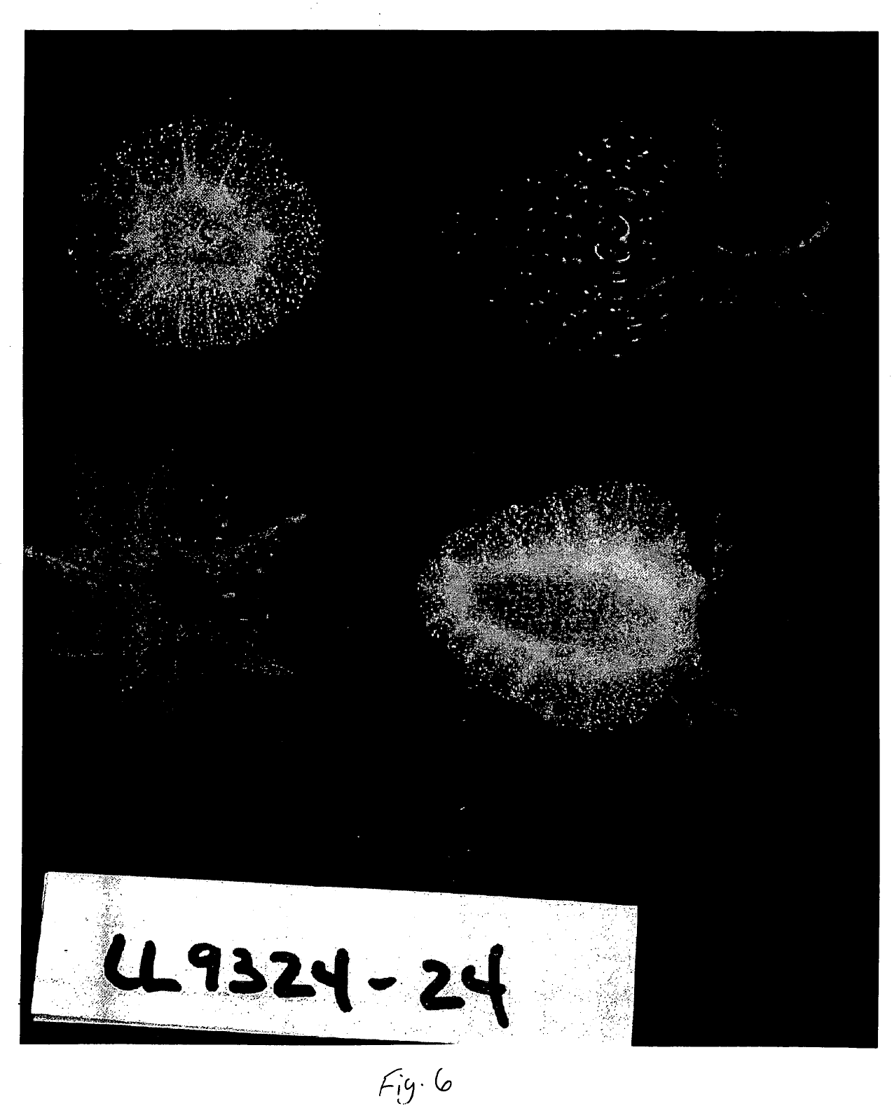

FIG. 7 illustrates a close-up of the fruits compared to Jewel.

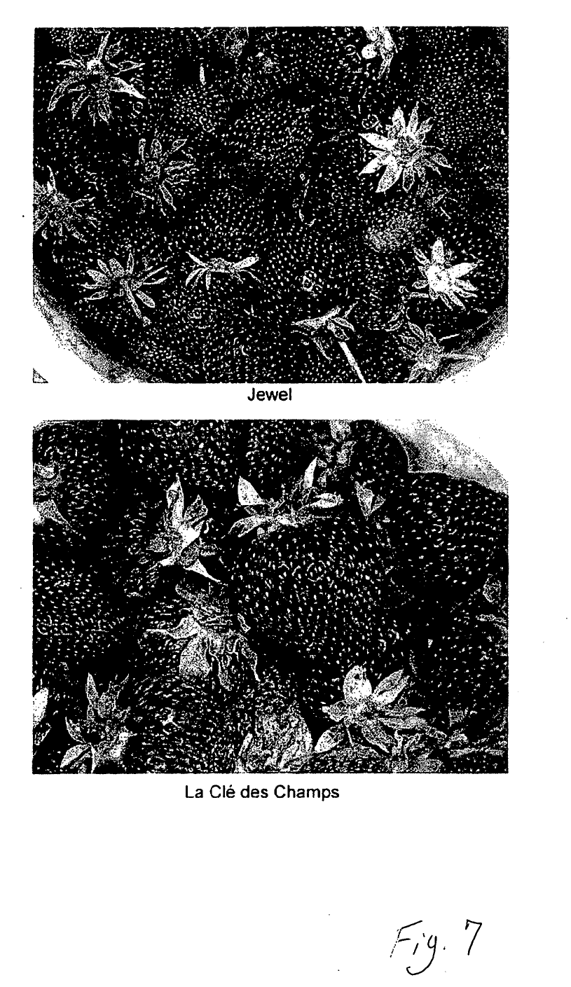

FIG. 8 illustrates the leaves of the plant compared to Jewel.

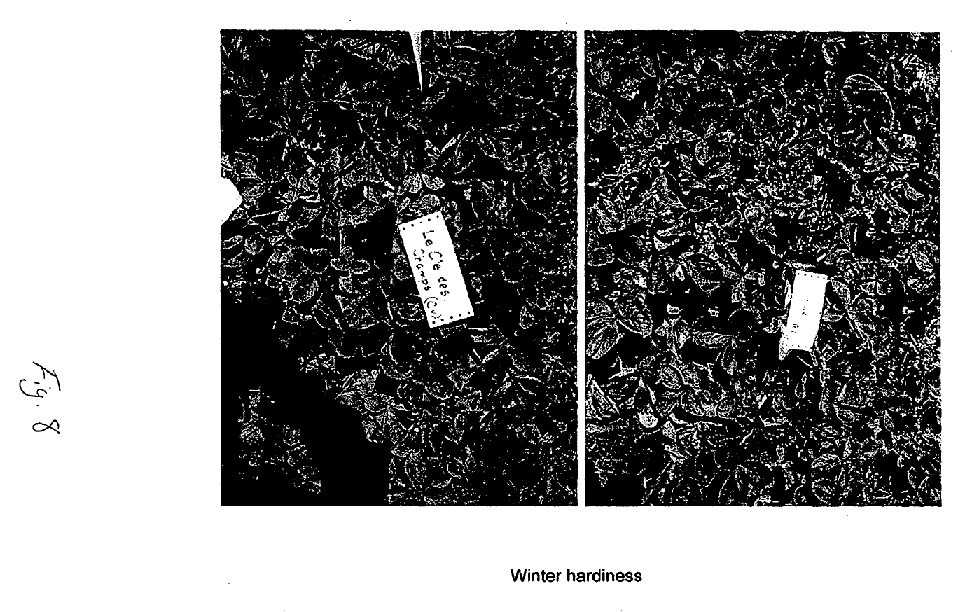

FIG. 9 illustrates the fruit in longitudinal and transversal cross-sections and upper side and side of the fruit compared to Jewel.

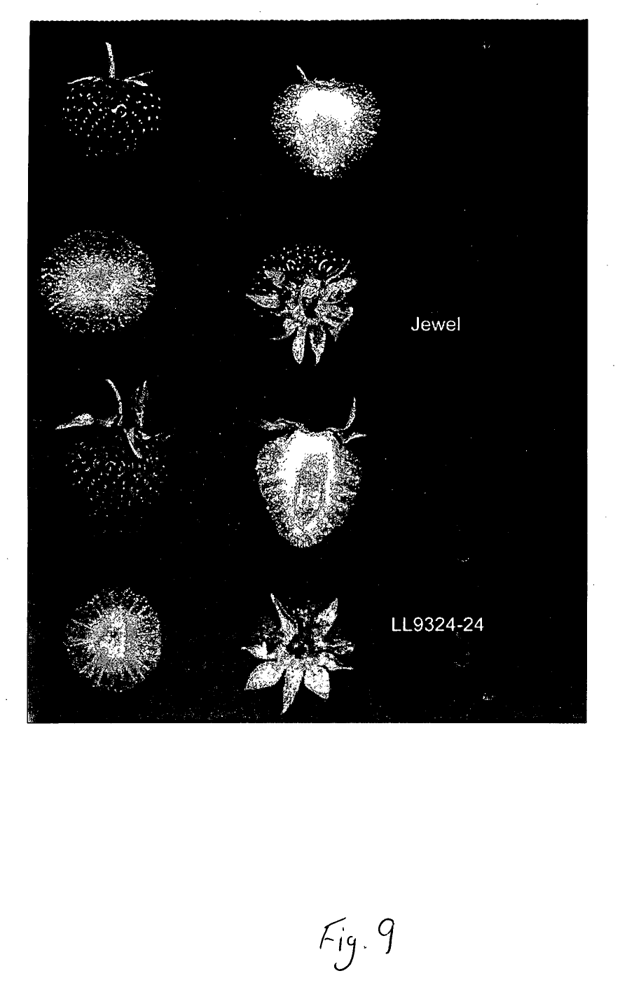

### DESCRIPTION OF THE NEW VARIETY

The following detailed description of the new variety is based upon observations taken of plants and fruit grown in l'Acadie, Québec.

Observations of “La Cle des Champs ” and “Jewel” were taken in side by side comparison in 2002-2003 growing season. This description is in accordance with UPOV terminology. Color designations, color descriptions, and other phonotypical descriptions may deviate from the stated values and descriptions depending upon variation in environmental, seasonal, climatic and cultural conditions. Colors are described and the most similar color designations are provided from The Royal Horticultural Society (R.H.S.) Colour Chart.

### PROPAGATION

The new variety is principally propagated by way of solons. Although propagation by solons is presently preferred, other known methods of propagating strawberry plants may be employed.

### CHARACTERISTICS OF THE NEW VARIETY

“La Cle des Champs” is a June-bearing strawberry variety with mid-season flowering and maturity. Plants are globose in habit, with strong vigour and high tolerance to low temperatures. The leaves are medium green with a slightly convex profile and weak blistering. There are three leaflets present. The terminal leaflet has a cupped profile and is slightly longer than broad. The terminal leaflet has an obtuse shaped base and the margin teeth are acute to obtuse in shape. The petiole has medium to dense pubescence with the hairs positioned outwards. “La Cle des Champs” produces many stolons which are medium to thick.

The medium to large flowers are positioned beneath to level with the foliage. The calyx is the same size to larger in diameter than the corolla and the inner calyx is smaller than to the same size as the outer calyx. The flower petals are touching to overlapping. The petal length/width ratio is broader than long.

The fruiting truss is semi-erect to prostrate and long. The fruit is longer than broad and ranges in size from medium to very large. The fruit is conical in shape with slight to moderate differences between the shape of the primary and secondary fruits. The achene band is medium to broad and the unevenness of the fruit surface is weak. “La Cle des Champs” has slightly uneven orange-red to red skin colour with strong glossiness. The achenes are inserted level with to above the fruit surface. The calyx is set above the fruit and the calyx segments are reflexed. The calyx is the same size as to larger than the fruit in diameter and adheres strongly to the fruit. The fruit flesh is firm to extremely firm with an uneven, orange red colour. The fruit has medium sweetness, acidity and texture. “La Cle des Champs” is resistant to powdery mildew and leaf scorch. It is moderately resistant to moderately susceptible to Verticillium wilt and leaf spots and moderately susceptible to all races of Red stele.

TABLE 1
Total yield, fruit weight and ripening season of ‘Clé des Champs’ and
comparison genotypes at the L'Acadie site.

| | Total yield^{z} | Wt./fruit^{z} | Ripening | |
|---|---|---|---|---|
| Genotype | (g · m⁻¹) | (g) | season^{y} | Hardiness |
| Kent | 2821.5a | 10.42b | EM | Very hardy |
| Clé des | 1633.3ab | 12.88a | M | Very hardy |
| Champs | | | | |
| Jewel | 1390.8b | 10.85ab | M | Less hardy |
| LSD | 1213.7 | 2.42 | | For details |
| | | | | see Fig of the |
| | | | | plots |

^{z}Averaged over 4 replicates from two field second year plantings (2003-2004), data taken from a 1 meter long representative portion of a 2 meter matted row (width 50 cm).
^{x}EM = Early midseason, M = midseason,

TABLE 2
Sugar, acidity, firmness, flavour, skin colour, leaf disease susceptibility,
SS and acidity of ‘Clé des Champs’ vs selected known cultivar
at L'Acadie.

| | | | | Leaf | | |
|---|---|---|---|---|---|---|
| | | | Skin | disease | | |
| Genotypes | Firmness^{y} | Flavour^{y} | colour^{y} | susceptibility | SS | Acidity |
| Jewel | 4.0 | 3.5 | 2.7 | Moderately | 7.9 | 0.81 |
| | | | | susceptible | | |
| Kent | 3.1 | 3.0 | 2.8 | Very | — | — |
| | | | | susceptible | | |
| Clé des | 4.0 | 3.5 | 2.7 | Moderately | 6.1 | 0.824 |
| Champs | | | | susceptible | | |
| LSD | | | | | | |

^{z}Averaged over 2 years from second year plantings (2003-2004), minimum of four replications per year, data taken from a 1 meter long representative portion of a 2 meter matted row (width 50 cm).
^{y}Firmness: 1 = very soft, 5 = very firm; flavour: 1 = poor, 5 = excellent; skin colour: 1 = very pale, 5 = dark red; leaf. See also Khanizadeh et al. (1994) for details on score evaluations

TABLE 3
Groups of phenolic compounds (ppm) and antioxidant property of Jewel vs
Clé des Champs grown at L'Acadie site in 2003.

| | | Hydroxy- | | | | |
|---|---|---|---|---|---|---|
| | Antho- | cinnamic | | Ellagic | Benzoic | |
| Genotype | cyanins | acids | Flavonols | acids | acids | Total |
| Clé des | 114.46b | 5.88fg | 4.48f | 2.63fg | 27.19d | 154.64b |
| Champs | | | | | | |
| Jewel | 140.21a | 4.34gh | 6.40^{e} | 2.62fg | 15.60g | 169.17a |
| LSD_{0.05} | 6.51 | 1.68 | 0.89 | 0.36 | 1.55 | 6.68 |

Groups of phenolic compounds were quantified as follows: ellagic acids as ellagic acid, benzoic acids as gallic acid, hydroxycinnamic acids as p-coumaric acid, flavonols as quercetin-3-galactoside, and anthocyanins as cyanidin-3-galactoside.

1. A new and distinct variety of strawberry plant, substantially as shown and described.
